(19) United States
(12) Patent Application Publication
(10) Pub. No.: US 2021/0307676 A1
(43) Pub. Date: Oct. 7, 2021

(54) NON-INVASIVE AND MINIMALLY-INVASIVE DETECTION OF SERUM IRON IN REAL TIME

(71) Applicant: CVDevices, LLC, San Diego, CA (US)

(72) Inventors: Ghassan S. Kassab, La Jolla, CA (US); Ali Dabiri, San Diego, CA (US)

(73) Assignee: CVDevices, LLC, San Diego, CA (US)

(21) Appl. No.: 17/354,497

(22) Filed: Jun. 22, 2021

Related U.S. Application Data
(63) Continuation-in-part of application No. 16/502,005, filed on Jul. 2, 2019.
(60) Provisional application No. 62/799,159, filed on Jan. 31, 2019.
(60) Provisional application No. 62/701,073, filed on Jul. 20, 2018.
(60) Provisional application No. 62/693,367, filed on Jul. 2, 2018.

(51) Int. Cl. A61B 5/00; A61B 10/00; G01N 1/34; G01N 11/02

(52) U.S. Cl. CPC A61B 5/449; A61B 10/0096; A61B 5/448; G01N 2011/0086; G01N 11/02; A61B 2562/0223; A61B 2560/0431; G01N 1/34

(57) ABSTRACT

A non-invasive and minimally-invasive system and method of detecting iron content in nail and hair samples to diagnose iron deficiency anemia. The system comprises a portable and ultra sensitive magnetometer. A sample of hair or nail is collected from the patient and placed in the magnetometer. The magnetometer is sufficiently sensitive to detect or otherwise indicate the iron content of the sample. Diagnosis can be made based on the detected iron level.

### PRIORITY

The present application is a continuation-in-part of, and claims the priority benefit of, U.S. patent application Ser. No. 16/502,005, filed Jul. 2, 2019 which is related to, and claims the priority benefit of U.S. Provisional Patent Application Ser. No. 62/799,159, filed Jan. 31, 2019, U.S. Provisional Patent Application Ser. No. 62/701,073, filed Jul. 20, 2018, and U.S. Provisional Patent Application Ser. No. 62/693,367, filed Jul. 2, 2018. The contents of each of these applications are incorporated into the present disclosure by reference in their entirety.

### BACKGROUND

Nearly two billion people and approximately 300 million children globally are afflicted with iron deficiency. Lack of iron causes anemia, impairs cognitive and behavioral development in childhood, compromises immune responsiveness, diminishes physical performance, and, when severe, increases mortality among infants, children, and pregnant women. Most of those affected are unaware of their lack of iron, in part because detection of iron deficiency requires a blood test. It is becoming increasingly important to screen these individuals to reduce medical cost and avoid chronic disease conditions. There are limited settings of laboratory infrastructure for standard blood-based tests around the World to accomplish this important screening test. Non-invasive screening is likely to be more acceptable to children and many other populations than methods requiring finger or vein puncture.

Presently, there are commercially available iron assay kits in the market. The disadvantage is the need for a blood sample and the time of the assay, which takes a minimum of one hour to perform. The kit measures iron in the linear range of 0.4 to 20 nmol in 50 μl sample. The assay produces a stable colored complex at 593 nm wavelength that can be detected with a photo detector.

In view of the same, there is a need for non-invasive and minimally invasive methods to provide a rapid, easy to use means for point-of-care (POC) screening for iron deficiency in resource-limited settings lacking laboratory infrastructure.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows components of an exemplary system of the present invention; and

FIG. 2 shows a diagram of an exemplary method of the present invention.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### BRIEF SUMMARY

The present disclosure includes disclosure of two different mechanisms to detect serum iron content in real time, namely non-invasive mechanisms/methods and minimally-invasive mechanisms/methods.

The present disclosure includes disclosure of a method for detecting serum iron content, comprising positioning a device relative to a nonpigmented epithelial layer covering capillaries of a mammalian subject, operating the device to obtain optical data relating to the capillaries, and determining serum iron content of blood within the capillaries based upon the optical data.

The present disclosure includes disclosure of a method further comprising the step of determining whether or not the mammalian subject is anemic based upon the determined serum iron content.

The present disclosure includes disclosure of a method, wherein the step of operating the device comprises operating a fluorescence spectroscopy device.

The present disclosure includes disclosure of a method, wherein the step of operating the device comprises operating the device to illuminate and acquire a fluorescence emission spectra from the subject.

The present disclosure includes disclosure of a method, wherein the step of operating the device comprises operating an optical fiber probe of the device to illuminate and acquire the fluorescence emission spectra from the subject.

The present disclosure includes disclosure of a method, wherein the step of operating the device comprises operating the device to obtain the optical data relating to the presence of zinc protoporphyrin of the blood.

The present disclosure includes disclosure of a method, wherein the step of positioning is performed by positioning the device relative to a lower lip of the mammalian subject.

The present disclosure includes disclosure of a method, wherein the step of operating the device comprises operating a terahertz spectroscopy device.

The present disclosure includes disclosure of a method, wherein the step of operating the device comprises operating the device to illuminate and acquire a terahertz emission spectra from the subject.

The present disclosure includes disclosure of a method, wherein the step of operating the device comprises operating an optical fiber probe of the device to illuminate and acquire the terahertz emission spectra from the subject.

The present disclosure includes disclosure of a method, wherein the step of operating the device comprises operating the device to obtain the optical data relating to an intensity of the terahertz emission spectra, whereby the intensity corresponds to a concentration of the serum iron content of the blood.

The present disclosure includes disclosure of a method for detecting serum iron content, comprising obtaining blood from a mammalian subject, operating a device to excite electrons within the blood and to measure a wavelength of emitted energy during a return of the excited electrons to a ground state, and determining serum iron content of the blood based upon wavelength of the emitted energy.

The present disclosure includes disclosure of a method, wherein the step of operating the device comprises operating an inductively coupled plasma atomic emission spectroscopy (ICP-AES) device.

The present disclosure includes disclosure of a method, wherein the step of operating the device comprises operating an inductively coupled plasma atomic optical spectroscopy (ICP-AOS) device.

The present disclosure includes disclosure of a method for detecting serum iron content, comprising obtaining blood from a mammalian subject, operating a device to obtain data relating to the blood, the data selected from the group consisting of viscosity data and conductance data, and determining serum iron content of the blood based upon the obtained data.

The present disclosure includes disclosure of a method, wherein the step of operating the device comprises operating a device configured to generate a magnetic field while obtaining the viscosity data.

The present disclosure includes disclosure of a method, wherein the step of operating the device comprises (a) obtaining first viscosity data relating to the blood using a device configured to obtain viscosity data, and (b) obtaining second viscosity data relating to the blood using the device configured to obtain viscosity data while a magnetic field is applied to the blood.

The present disclosure includes disclosure of a method, wherein the step of determining serum iron content is performed by comparing the first viscosity data to the second viscosity data.

The present disclosure includes disclosure of a method, wherein the step of operating the device comprises operating a device configured obtain the conductance data.

The present disclosure includes disclosure of a method, wherein the obtained data comprises the conductance data, whereby relatively low conductance data is indicative of low serum iron content.

The present disclosure includes a method for detecting iron content in nail or hair, comprising: collecting a sample of nail or hair from a patient; positioning the collected sample in a portable and ultra sensitive magnetometer; and activating the magnetometer to detect iron in the sample.

In a further embodiment, the magnetometer has sensitivity such that it can detect 0.1 μg of pure iron or a signal of about 1 μemu and a noise level around 20 nemu. In another embodiment, the magnetometer has a dynamic range of about 20.

In a further embodiment of a method for detecting iron content in a nail or hair sample using a magnetometer, the sample is a sample of hair. In a further embodiment, the samples is a sample of nail wherein the sample of nail weighs at least 50 mg.

In a further embodiment of a method for detecting iron content in a nail or hair sample using a magnetometer, the sample is cleaned. In a further embodiment, the step of cleaning the sample comprises acid washing the sample to remove surface iron contamination. The step of cleaning the sample may be repeated three times. The step of cleaning is followed by a step of drying the sample.

In an alternate embodiment of a method for detecting iron content in a sample of hair or nail wherein a sample of hair or nail is placed into an ultra-sensitive magnetometer and the magnetometer is configured to indicate the amount of iron present in the sample.

In a further embodiment of a method for detecting iron content in a nail or hair sample using a magnetometer, the sample of nail weighs about 50 mg.

In a further embodiment of a method for detecting iron content in a nail or hair sample using a magnetometer, the magnetometer is configured to detect the iron present in a 50 mg sample of nail.

In a further embodiment of a method for detecting iron content in a nail or hair sample using a magnetometer, the sample is free from surface iron contamination

In a further embodiment of a method for detecting iron content in a nail or hair sample using a magnetometer the sample is cleaned to remove surface iron contamination before positioning the sample in the magnetometer. In an alternate embodiment, the sample is cleaned with acid and then dried.

In a further embodiment of a method for detecting iron content in a nail or hair sample using a magnetometer, the magnetometer is configured to distinguish a sample having less than 10 μemu.

In an embodiment of an ultra-sensitive and portable magnetometer configured to detect iron human nail or hair samples, the magnetometer comprises: a detection limit of about 0.1 μg for pure iron corresponding to a signal level as low as 1 μemu and a noise level around 20 nemu; and a dynamic range of about 20-30.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure of two different mechanisms to detect serum iron content in real time, namely non-invasive mechanisms/methods and minimally-invasive mechanisms/methods.

Devices, systems, and methods to detect iron present in hair and nail are also described.

### Non-Invasive Methods

The use of a patient's hair or nails (fingernails or toenails) to estimate the serum iron content is referenced herein. The possibility of an optical signature of the serum iron can also be explored if the goal is a non-invasive and real time assay. The present disclosure considers two optical approaches, namely florescence spectroscopy and Tera-Hertz (THz) spectroscopy. In these methods, the serum iron needs to have signature with very high sensitivity and specificity and the response needs to be linear with the concentration of the serum iron.

### Iron Content in Hair/Nail

Human serum is recognized as the “gold standard” to determine iron and other mineral levels. It is important to note that in the most widely-used test of serum ferritin level, the body iron status may not be accurately reflected due to various conditions, including pregnancy, acute or chronic inflammatory disease, malignancy, infection, renal failure, or malabsorption syndrome. Hair can be an attractive alternative due to its simplicity as a sample (easy to obtain, without trauma and/or discomfort), storage, transport and handling.

The determination of hair iron concentration necessitates a strict sampling regime, however, which is not practical. Historically, there has been little data associated with the use of hair iron concentration to define body iron status. In 1956, Duffield et al. concluded that hair iron concentration may not provide sufficient information regarding total body iron. In 1971, Lovric et al. measured the iron content of various hair segments of children with iron deficiency and iron overload and concluded that there was no significant association between the groups with respect to hair iron concentration. In subsequent years, however, Bisse et al. concluded that hair iron concentration is useful in the evaluation of body iron status. Sahin et al. studied the possible association between blood parameters and hair iron concentration in patient groups with different body iron contents through chemical analysis. The study population comprised of 25 patients (mean of 33 years) with iron deficiency anemia and 20 patients (mean of 22 years) with transfusion-related anemia that showed a difference in body iron content. The 21 healthy control group was formed of age (mean of 28 years) and gender-matched subjects with no history of underlying disease. The results showed measured mean hair iron ⁵⁶Fe and ⁵⁷Fe concentrations of the iron deficiency group were 5.08 and 6.03 μg/g, respectively, and in the transfusion-related anemia group these values were 28.9 and 29.4 μg/g, respectively. In the control group, the mean hair iron ⁵⁶Fe and ⁵⁷Fe concentrations were measured as 12.0 and 17.6 μg/g, respectively. The highest hair iron concentration (89.4 μg/g) was observed in transfusion-related anemia patients, whereas the lowest hair iron concentration (0.77 μg/g) was determined in the iron deficiency anemia group. The differences between the three groups with respect to hair iron ⁵⁶Fe and ⁵⁷Fe concentrations were found to be statistically significant. In addition, a positive correlation was determined between hair iron ⁵⁶Fe and ⁵⁷Fe concentrations and serum iron, ferritin level, transferrin saturation, MCV and MCH values, which are the most important parameters showing body iron content. This study concluded that patient groups with different body iron content had a significant difference in hair iron concentration and these values were correlated with laboratory markers of body iron content. These results support the view that hair sampling can be used as a marker of body iron content.

In another study, Claudio et al. developed a method to determine iron in human hair samples by graphite furnace atomic absorption spectrometry (GF AAS). They measured iron levels in hair samples from 20 pre-adolescent, menstruating girls in schools in Brazil. The concentration range was 14-26 μg/g. Baranowska et al. analyzed hair samples collected from the inhabitants of Poland by x-ray fluorescence spectrometry and obtained an average concentration of 36.3 μg/g for Fe in hair samples.

Human nail (fingernails and toenails) can also be an attractive alternative due to its simplicity as a sample (easy to obtain, without trauma and/or discomfort), storage, transport and handling. Sobolewski et al. measured the iron content of healthy and iron deficient individual nails. The iron content of the nails ranged from 6 to 26 μg/g of nail for the women and 6 to 23 μg/g for the men in healthy individual group. This value dropped to less than 4 μg/g for the iron deficient subjects. In iron-depleted and iron-sufficient subjects there was a correspondence between iron content of the nails and bone marrow iron, serum iron and TIBC.

The major disadvantage of these methods is the need to transport the hair/nail samples to an analytical laboratory for testing which is time consuming, expensive and the facility may not be accessible in developing countries.

### Detection of Iron Content in Hair/Nail by Magnetometer

The iron in hair and nail is detectable using a magnetometer. One example of a magnetometer can be vibrating sample magnetometer (VSM). A VSM is a scientific instrument that measures magnetic properties. Simon Foner at MIT Lincoln Laboratory invented VSM in 1955 and reported it in 1959. A sample is first magnetized in a uniform magnetic field. It is then sinusoidally vibrated, typically through the use of a voice coil actuator. The induced voltage in the pickup coil is proportional to the sample's magnetic moment, but does not depend on the strength of the applied magnetic field. In a typical setup, the induced voltage is measured with a lock-in amplifier using the vibration frequency as the reference.

The density of human nail is ˜0.65 g/cc (as measured in the inventors' lab). The average nail growth is ˜0.8 mm/week which translates into 5 mg/week for an average finger or 50 mg/week for ten fingers. The average iron concentration in human nail for an iron deficient anemic patient is about 1 μg/g according to previous research (but can vary from about 1-2 μg/g). One gram of nail from an iron deficiency anemia patient should have a magnetic moment of 1 μg×210 emu/g=0.0002 emu or ˜200 μemu. Therefore, the magnetic moment for a week of nail growth is about 10 μemu, assuming the iron is in the form of pure iron. Iron content in nails is about 26 μg/g for a normal non-iron deficient human.

The iron content in hair samples is also relatively small. As described above, concentrations of ⁵⁶Fe and ⁵⁷Fe in hair samples in iron deficiency anemia patients average 5.08 and 6.03 μg/g, respectively. The magnetic moment of one gram of hair in an iron deficient anemia patent should then be approximately 2333 μemu. Levels of ⁵⁶Fe and ⁵⁷Fe in normal patients were measured as 12.0 and 17.6 μg/g, respectively.

The present disclosure describes novel devices, systems and methods of using a magnetometer to detect iron content in hair or nail to screen for iron deficient patients. Embodiments herein take advantage of the ease of hair and nail sampling, while solving the problems associated with such sampling, such as the need to transport and evaluate samples in a laboratory setting. The processes herein can be performed outside of a laboratory or hospital setting and with minimal supplies, thus enabling patient care where access to medical care or laboratory services is difficult to obtain. Furthermore, as the exemplary magnetometers are portable, no transportation of sample is required and the entire process of testing can be performed on-site. The process enables real time response and provides point of care screening. In addition, the testing is non-invasive and does not require chemicals during the sample analysis.

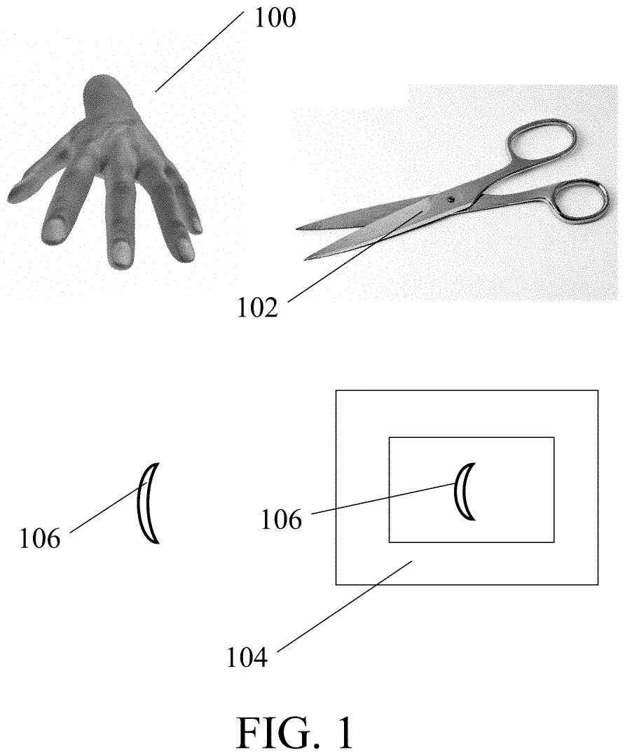

FIG. 1 illustrates the components comprising the disclosed system for testing a nail sample for iron content. It is understood that hair samples can be tested using the same devices, systems and methods as described herein for nail samples. Thus, in FIG. 1, the nail sample 106 would be replaced with a hair sample when testing hair.

In an embodiment, an exemplary magnetometer 104 of the present invention is a portable unit that can be operated with a trained technician. Magnetometers of the present invention are configured to be able to identify the relatively small iron concentrations in hair and nail samples. As the normal iron content of hair and nail is already relatively small and iron deficient patients have an even smaller amount of iron in their hair and nails, an ultra-sensitive magnetometer would be utilized to analyze samples. Thus, in the exemplary embodiment the magnetometer has a detection limit of about 0.1 μg for pure iron, corresponding to a signal level as low as 1 μemu and a noise level around 20 nemu. The magnetometer should have a dynamic range of about 20. A dynamic range of 30 for design considerations can also be considered.

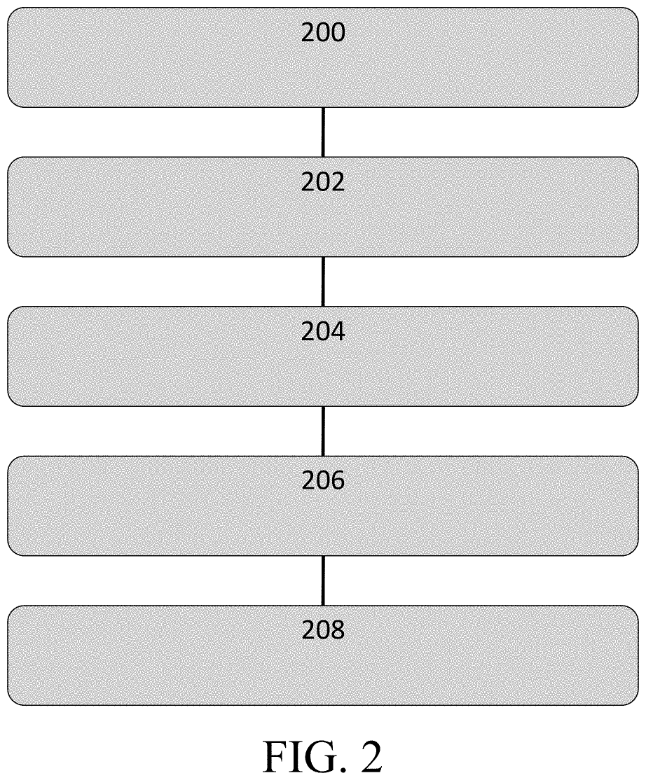

FIG. 2 shows a diagrammatic view of an exemplary embodiment of a method of testing for iron deficiency using a portable and ultra sensitive magnetometer. In collection step 200, a sample of hair or nail is taken from a patient. The samples should be obtained from the patient 100 using stainless steel scissors or clippers, or a suitable device 102. While stainless steel has much weaker magnetic properties than iron, any steel contamination may cause error in measurement. Where the sample is nail, the sample preferably weighs at approximately or at least 50 mg. The sample collected is preferably stored in an auto-sealable polythene bag and may be acid washed in a cleaning step 202.

Any coatings may interact with the surface atoms of the magnetic core and form a magnetically disordered layer, reducing the total amount of the magnetic phase. A common contaminant is nail polish. Prior to the nail sampling process, there should not be any cosmetic nail applications, such as said nail polish. If needed, contamination may be removed by cleaning.

In the cleaning step 202, the hair/nail sample 106 is prepared in such a manner to eliminate all possible environmental contaminations such as iron or other magnetic materials. Cleaning can be done with an acid wash (such as with nitric and hydrochloric acid) to remove contamination. The cleaning can be repeated, such as three times, to ensure no residual contamination. Less harsh cleaning solutions may also be used. Where the cleaning solution is not toxic/harmful, cleaning step 202 can be performed before collection step 200. For example, hair can be washed with shampoo. After cleaning, the samples should be appropriately dried, such as in an oven, before mounting it in the VSM sample holder.

The dried samples should then and mounted or otherwise placed in the magnetometer configured to detect iron and having sensitivity as described above, in mounting step 204. The magnetometer is then activated and the iron content assessed in detection step 206. Depending on the results of the magnetometer analysis, the patient can then be diagnosed as iron deficient or not iron deficient in diagnosis step 208.

### Florescence Spectroscopy

In the developing red blood cell, the insertion of iron into protoporphyrin IX is the final step in the production of haem for incorporation into haemoglobin. If iron is unavailable, divalent zinc is incorporated instead, producing zinc protoporphyrin, which persists for the life of the red blood cell as a biochemical indicator of functional iron deficiency. In regions with endemic for malaria and other infections, the World Health Organization recommends measurement of the red blood cell zinc protoporphyrin as the preferred indicator to screen children for iron deficiency. In the United States, the American Academy of Pediatrics recommends universal screening for iron deficiency at one year of age, and the use of red blood cell zinc protoporphyrin for this purpose has been suggested. Screening for iron deficiency using red blood cell zinc protoporphyrin has recently been proposed as standards. With blue light excitation, zinc protoporphyrin fluoresces, while haem does not. The feasibility to detect this fluorescence is included in the present disclosure, where an optical fiber probe can be used to illuminate and acquire the fluorescence emission spectra from the lower lip, where only a thin, nonpigmented epithelial layer covers the blood-filled capillaries perfusing the underlying tissue. A portable fluorescence spectroscopy device would be ideal for use in regions where medical facilities are not readily available or accessible.

### Terahertz (THz) Spectroscopy

THz spectroscopy and imaging (imaging at frequencies around 10¹² Hz) is a novel technique for medical imaging. It uses non-ionizing radiation and can safely be used for imaging different types of tissue, such as normal cells and tumors; the contrast between tissue types is thought to occur due to differences in water content, protein density or cellular structure. Penetration of tissue depends on the fat and water content and can reach a depth ranging from several hundred microns to several millimeters.

Terahertz spectroscopy has been used to characterize the blood. The complex optical constants of blood and its constituents, such as water, plasma, and red blood cells (RBCs), were obtained in the THz frequency region. The volume percentage of RBCs in blood was extracted and compared with the conventional RBC counter results. The THz absorption constants are shown to vary linearly with the RBC concentration in both normal saline and whole blood. The feasibility of this technique is referenced herein to detect the iron deficiency and its sensitivity and specificity. An optical fiber probe is used to illuminate and acquire the terahertz emission spectra from the lower lip, where only a thin, nonpigmented epithelial layer covers the blood-filled capillaries perfusing the underlying tissue. The rationale is that the optical signature intensity is proportional to the concentration of the RBC iron concentration. A portable THz spectroscopy device would be ideal for use in regions where medical facilities are not readily available or accessible.

### Minimally-Invasive Methods

Small blood samples are necessary for in vitro analysis, as referenced herein. Three methods, namely Inductively Coupled Plasma Atomic Emission (or Optical) Spectroscopy (ICP-AES, or ICP-AOS), serum viscosity change in a magnetic field, and bio-impedance are disclosed herein.

Traditionally, serum would need to be separated from the blood in order to measure the iron level in blood due to transferrin, which is one of three markers doctors usually order to find the status of the iron in the body (the other two are TIBC and ferritin). In other situations, such as regions with endemics for malaria and other infections, the World Health Organization (WHO) recommends measurement of the red blood cell zinc protoporphyrin as the preferred indicator to screen children for iron deficiency.

In the methods noted below, blood samples can be used directly rather than serum.

### ICP-AES/ICP-AOS

ICP-AES/ICP-AOS are emission spectrophotometric techniques, exploiting the fact that excited electrons emit energy at a given wavelength as they return to a ground state after excitation by high temperature argon plasma. The rationale of this process is that each element emits energy at specific wavelengths peculiar to its atomic character. The energy transfer for electrons when they fall back to the ground state is unique to each element as it depends upon the electronic configuration of the orbital. This technique has been used to analyze biological samples. The analysis can be made in real time with high detection sensitivity. The unit size is tabletop, although some portable systems have been built for metallic element analysis in the warehouses. This technique can be utilized to detect serum iron and its sensitivity with different blood samples. Once satisfied, the unit can be tailored for this purpose and make it smaller for the bed-side application.

### Assays in Magnetic Fields

Physicists Rongjia Tao and Ke Huang took donated blood and then measured its viscosity in a small tube used for that purpose. They then applied a 1.3 Tesla magnetic field to the tube (this is about the strength of the magnetic field used in a typical MRI scanner), with the field aligned with the direction of blood flow, for one minute and found that the viscosity decreased by 20-30%. This effect lasted for about 2 hours. The rationale comes from the blood cells clumping together, mostly in a line, like box cars on a train. The cells moving together as a train produces less resistance than if they were all bouncing around separately. Further, they tend to flow more down the middle of the tube, reducing friction with the tube wall. The glass tube used in the study was larger than the smallest arteries in humans. It is postulated that the viscosity in this set-up is directly proportional to the iron content of the RBC in the blood. This method can be used to determine the iron deficiency of the blood. This concept, as noted in the present disclosure, can be used to measure the iron content in the serum in a magnetic field if the interest is the measurement if the iron in the serum. The change of viscosity can be measured by a viscometer. The magnet with the 1.3 T strength can be rather small since the core of the magnet where the sample is placed can be as small as 0.5 cm in diameter. The best candidate is neodymium magnets.

Neodymium magnets, invented in the 1980s, are the strongest and most affordable type of rare-earth magnet. They are made of an alloy of neodymium, iron, and boron (Nd₂Fe₁₄B), sometimes abbreviated as NIB. Neodymium magnets are used in numerous applications requiring strong, compact permanent magnets, such as electric motors for cordless tools, and hard disk drives. They have the highest magnetic field strength and have a higher coercivity (which makes them magnetically stable). Since their prices became competitive in the 1990s, neodymium magnets have been replacing ferrite magnets in the many applications in modern technology requiring powerful magnets. Their greater strength allows smaller and lighter magnets to be used for a given application. The speakers use this kind of magnets with about 1.4 T magnetic strength and the sizes are not big by any standard.

### Bio-Impedance

A bio-impedance method can also be used to detect iron levels in real time. Iron is electrically conductive, and the concentration of iron is proportional to electrical conductance (inverse of impedance); i.e., less iron implies lower electrical conductance. As such, operating a conductance device on a blood sample can result in obtaining conductance data, and relatively low conductance data is indicative of low iron concentration.

While various embodiments of methods and devices for the non-invasive detection of serum iron in real time have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

### REFERENCES

1. Sahin C. et al., Measurement of hair iron concentration as a marker of body iron content, Biomedical reports, Volume 3, Issue 3, DOI: 10.3892/br.2015.419, 2015.
2. Duffield J and Green P T: The iron content of human hair. II. Individuals with disturbed iron metabolism. Can Serv Med J 12: 987-996, 1956.
3. Lovric V A and Pepper R: Iron content of hair in children in various states of iron balance. Pathology 3: 251-256, 1971.
4. Bisse E, Renner F, Sussmann S, Scholmerich J and Wieland H: Hair iron content: possible marker to complement monitoring therapy of iron deficiency in patients with chronic inflammatory bowel diseases, Clin Chem 42: 1270-1274, 1996.
5. Cláudio L. Donnici et al. Fast Determination of Iron and Zinc in Hair and Human Serum Samples After Alkaline Solubilization by GF AAS, J. Braz. Chem. Soc., Vol. 27, No. 1, 119-126, 2016.
6. Baranowska, I.; Barchanski, L.; Bak, M.; Smolec, B.; Mzyk, Z.; Pol. J. Environ. Stud., 13, 369, 2004.
7. Sobolewski, S. et al., Human nails and body iron, j. Clinical Pathology, 31, 1068-1072, 1978.
8. Wikipedia, VSM, accessed in July 2019.

1. A method for detecting iron content in nail or hair, comprising:
collecting a sample of nail or hair from a patient;
positioning the collected sample in a portable and ultra sensitive magnetometer;
activating the magnetometer to indicate the amount of iron in the sample.

2. The method of claim 1, wherein the magnetometer has a sensitivity such that it can detect 0.1 μg of iron or a signal of about 1 μemu and a noise level around 20 nemu.

3. The method of claim 2, wherein the wherein the magnetometer has a dynamic range of about 20-30.

4. The method of claim 1, wherein the sample is a sample of nail.

5. The method of claim 4, wherein the sample of nail weighs at least 50 mg.

6. The method of claim 1, wherein the sample is a sample of hair.

7. The method of claim 1, further comprising the step of cleaning the sample.

8. The method of claim 7, wherein the step of cleaning the sample comprises acid washing the sample to remove surface iron contamination.

9. The method of claim 8, further comprising the step of drying the sample.

10. The method of claim 9, wherein the step of cleaning the sample is repeated three times before drying.

11. A method for detecting iron content in a sample of hair or nail comprising the steps of:
placing a sample of hair or nail obtained from a patient into an ultra-sensitive magnetometer; and
activating the magnetometer wherein the magnetometer indicates the level of iron present in the sample of hair or nail wherein the level of iron is indicative of iron deficiency anemia in the patient.

12. The method of claim 11, wherein the sample is nail and weighs about 50 mg.

13. The method of claim 11, wherein the magnetometer is configured to detect the level of iron present in a 50 mg sample of nail.

14. The method of claim 11, wherein the magnetometer has a sensitivity such that it can detect 0.1 μg of iron or a signal of about 1 μemu and a noise level around 20 nemu.

15. The method of claim 14, wherein the magnetometer has a dynamic range of about 20-30.

16. The method of claim 11, wherein the sample is free from surface iron contamination

17. The method of claim 14, wherein the sample is cleaned to remove surface iron contamination before positioning the sample in the magnetometer.

18. The method of claim 17, wherein the sample is cleaned with acid and then dried.

19. The method of claim 11, wherein the magnetometer is configured to detect a magnetic moment of 10 μemu.

20. An ultra-sensitive and portable magnetometer configured to detect iron human nail or hair samples comprising:
a detection limit of about 0.1 μg for iron corresponding to a signal level as low as 1 μemu and a noise level around 20 nemu; and
a dynamic range of about 20.
